(19) United States
(12) Patent Application Publication
(10) Pub. No.: US 2026/0151563 A1
(43) Pub. Date: Jun. 4, 2026

(54) SAFETY NEEDLE ASSEMBLY

(71) Applicant: PROMISEMED HANGZHOU MEDITECH CO.,LTD., Hangzhou City, Zhejiang (CN)

(72) Inventors: Yan LU, Hangzhou City, Zhejiang (CN); Chaoyu HU, Hangzhou City, Zhejiang (CN)

(21) Appl. No.: 19/099,238

(22) PCT Filed: Mar. 8, 2023
(86) PCT No.: PCT/CN2023/080209

§ 371 Date: Feb. 6, 2026

(30) Foreign Application Priority Data
Jul. 29, 2022 (CN) 202210906666.8

(51) Int. Cl. A61M 5/158

(52) U.S. Cl. CPC A61M 5/158; A61M 2005/1586

(57) ABSTRACT

A safety needle assembly includes a needle seat, a needle tubing, a base and a connecting member. A guide hole for receiving the longitudinal tube to pass through is formed in one side of the base, and a receiving cavity is formed below the guide hole. The connecting member includes a connecting base and a foldable connector. When in the folded state, the longitudinal tube passes through the guide hole, the receiving cavity, and the connecting hole in sequence; and in the unfolded state, the tip of the longitudinal tube is located in the receiving cavity, and the connecting base rotates until the connecting hole and the longitudinal tube are in a staggered state. The tip of the needle tubing cannot enter the connecting hole again, so that the tip is restricted in the receiving cavity to play a protecting role in preventing medical staff from being stabbed.

### CROSS REFERENCE TO RELATED APPLICATIONS

This application is a national stage application under 35 U.S.C. § 371 of International Application No. PCT/CN 2023/080209 filed on Mar. 8, 2023, which claims priority under 35 U.S.C. § 119 to Application No. 202210906666.8 filed in China on Jul. 29, 2022; the entire contents of both of which are hereby incorporated by reference.

### TECHNICAL FIELD

The present disclosure relates to the technical field of medical apparatus and instruments, and in particular, to a safety needle assembly.

### BACKGROUND

A special-purpose needle for an implantable drug-deliverying device is called a Huber safety needle for short. The Huber safety needle is mainly used as an accessory of deep venous port access and is usually used together with an implantation port. Such Huber safety needle performs operations such as chemotherapy and intravenous injection through the implantation port. An implantation section is a self-sealed diaphragm for fixing the Huber needle and sealing the periphery of the Huber needle. An implantation end can realize multiple times of punctures without being damaged.

### TECHNICAL PROBLEM

Conventional Huber needle brings convenience to patients requiring subcutaneous injection, but may bring a certain danger to medical staff. It is usually needed to pull out the conventional Huber needle from the implantation port with both hands, with one hand fixing the implantation end and the other hand pulling out the needle. The force applied to pull out the needle from the self-sealed diaphragm of the implantation port may induce a bounce of the needle and stab the user. This may cause transmission of hematogenous pathogens to the user and may even make the user contact with dangerous drugs.

The U.S. Pat. No. 10,363,376B2 has disclosed a safety device for protecting a medical needle, which includes a handle element for connecting a Huber needle, a pedestal, and a connecting structure connecting the handle element and the pedestal, where the connecting structure is foldable. After injection is completed, the handle element and the Huber needle are pulled to move away from the pedestal until the connecting structure is unfolded, a syringe needle is hidden in the pedestal, so that the risk that the conventional Huber needle may stab medical staff is avoided. However, in the structure disclosed in the above patent, the mechanism for protecting the syringe needle is relatively complex, resulting in higher costs and to-be-improved reliability of the safety device.

### SUMMARY

The present disclosure aims to provide a safety needle assembly with an improved structure to overcome the deficiency in the prior art.

To solve the above technical problem, the present disclosure provides the following technical solution: a safety needle assembly, which at least includes:

a needle seat;

a needle tubing, where the needle tubing is bent to form a horizontal tube and a longitudinal tube, and the horizontal tube is fixedly connected with the needle seat;

a base, where a guide hole for receiving the longitudinal tube to pass through is formed in one side of the base facing the needle seat, and a receiving cavity is formed below the guide hole; and

a connecting member, where the connecting member includes a connecting base and a foldable connector, the connecting base is located below the receiving cavity and is rotationally and movably connected with the base, one end of the connector is connected with the connecting base and the other end of the connector is connected with the needle seat; and a connecting hole for receiving the longitudinal tube to pass through is formed in the connecting base.

The connector includes a folded state and an unfolded state. In the folded state, the longitudinal tube passes through the guide hole, the receiving cavity, and the connecting hole in sequence; and in the unfolded state, the tip of the longitudinal tube is located in the receiving cavity, and the connecting base rotates until the connecting hole and the longitudinal tube are in a staggered state.

In a preferred embodiment, a non-return flange is arranged on one side of the connecting base, and a non-return retaining arm adapted to the non-return flange is arranged on one side of the receiving cavity.

In a preferred embodiment, a gap is arranged on one side of the non-return retaining arm away from the connecting base.

In a preferred embodiment, the connector includes at least two connecting rods, and flexible portions are arranged between the needle seat and the connecting rod, between adjacent connecting rods, and between the connecting rod and the connecting base.

In a preferred embodiment, hinge pins are arranged at both ends of the connecting base, and pin holes adapted to the hinge pins are formed in an inner wall of the receiving cavity of the base.

In a preferred embodiment, a clamping mechanism is arranged between the needle seat and the base, and when the connector is in the folded state, the needle seat and the base are connected together through the clamping mechanism.

In a preferred embodiment, the clamping mechanism includes:

a pair of clamping arms extending toward one side of the base from the bottom of the needle seat;

protrusions protruding outward from the ends of free ends of the clamping arms; and

a receiving groove arranged on one side of the base facing the needle seat, where clamping edges adapted to the protrusions are arranged on groove walls of both sides of the receiving groove.

In a preferred embodiment, wings are arranged on both sides of the needle seat.

In a preferred embodiment, a bendable portion is arranged between the wing and the needle seat, and a damping portion is arranged on one side of the wing facing the base.

In a preferred embodiment, positioning bonding structures are arranged between the two wings, which include a positioning bonding protrusion and a positioning bonding groove adapted to each other.

According to the safety needle assembly in the embodiment, the connecting hole for receiving the longitudinal tube to pass through is formed in the connecting base. When the connector is in the folded state, the longitudinal tube passes through the guide hole, the receiving cavity, and the connecting hole in sequence. When the connector is in the unfolded state, the tip of the longitudinal tube is located in the receiving cavity. Since the connecting base rotates until the connecting hole and the longitudinal tube are in a staggered state, the tip of the needle tubing cannot enter the connecting hole again, so that the tip is restricted in the receiving cavity to play a protecting role in preventing medical staff from being stabbed.

According to the safety needle assembly with the above structure, by arranging the connecting base rotationally connected with the base and the structure of the connecting hole in the connecting base, the needle tip is protected. Compared with the prior art, the safety needle assembly has the technical advantages of a simpler structure, reliable protection, and lower costs.

### BRIEF DESCRIPTION OF THE DRAWINGS

FIG. 1 is a schematic structural diagram of a safety needle assembly in the embodiment, where a connector is in a folded state;

FIG. 2 is a schematic cross-sectional structural diagram of the safety needle assembly shown in FIG. 1;

FIG. 3 is a schematic structural diagram of the safety needle assembly shown in FIG. 1 sectioned at a clamping mechanism;

FIG. 4 is a schematic structural diagram of the safety needle assembly in the embodiment, where the connector is in an unfolded state;

FIG. 5 is a partial cross-sectional diagram of the safety needle assembly shown in FIG. 4 sectioned at a base;

FIG. 6 is a schematic structural diagram of the safety needle assembly in the embodiment in a changing process from a state shown in FIG. 1 to a state shown in FIG. 4;

FIG. 7 is a schematic structural diagram of a needle seat and a connecting member in the safety needle assembly in the embodiment, where the connector is in the unfolded state;

FIG. 8 is a schematic back diagram of the structure shown in FIG. 7;

FIG. 9 is a schematic structural diagram of the base in the safety needle assembly in the embodiment;

FIG. 10 is a schematic structural diagram of the base shown in FIG. 9 at an elevation angle;

FIG. 11 is a schematic cross-sectional structural diagram of the base shown in FIG. 9; and

FIG. 12 is a schematic three-dimensional state diagram of the cross-sectional structure shown in FIG. 11.

### DETAILED DESCRIPTION

For the purpose of making the objectives, technical schemes, and advantages of the present disclosure more clear, further detailed description will be made to the present disclosure in conjunction with the drawings and embodiments. It should be understood that the described specific embodiments are merely used to describe the present disclosure rather than to limit the present disclosure.

In the description of the present disclosure, it should be understood that, the orientation or positional relationship indicated by the terms “upper”, “lower”, “front”, “rear”, “inner”, “outer” and the like is based on the orientation or positional relationship shown in the accompanying drawings. This is only for the convenience of describing the present disclosure and simplifying the description, and does not indicate or imply that a device or element needs to have a specific orientation, or be constructed and operated in a specific orientation, and therefore cannot be understood as a limitation of the present disclosure.

In the description of the present disclosure, it should be noted that unless otherwise specified and defined, the terms “mounting”, “connecting”, and “connection” should be understood in a broad sense. For example, it may be fixed connection, integrated connection, or detachable connection; may be communication between two elements inside; and may be direct connection or indirect connection through an intermediate. Those of ordinary skill in the art may understand specific meaning of the terms in the present disclosure according to specific circumstances.

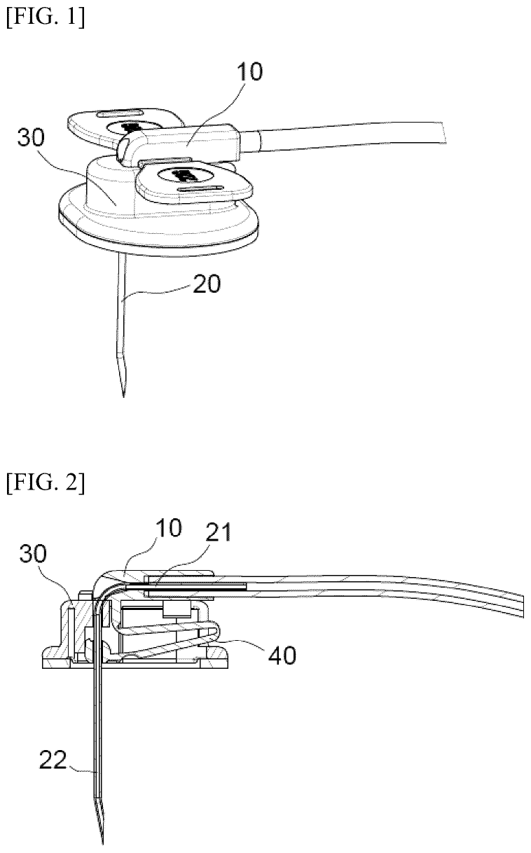

A safety needle assembly in the embodiment is a Huber safety needle. As shown in FIG. 1 to FIG. 4, the safety needle assembly includes a needle seat 10, a needle tubing 20, a base 30, and a connecting member 40.

The needle tubing 20 is bent to form a horizontal tube 21 and a longitudinal tube 22, the horizontal tube 21 is fixedly connected with the needle seat 10, and the longitudinal tube 22 extends toward one side of the base 30.

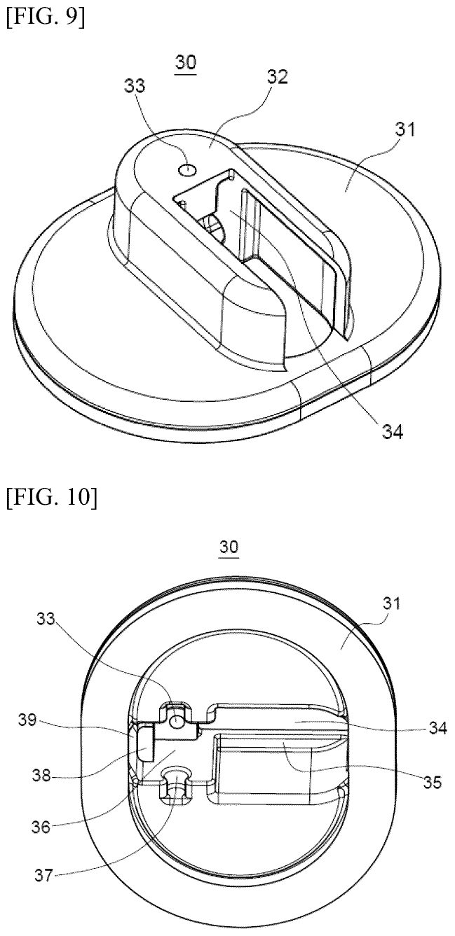

As shown in FIG. 9 to FIG. 12, the base 30 in the embodiment includes an annular pedestal 31 and a basal body 32 arranged on the pedestal 31. Preferably, an application layer 50 may be arranged at the bottom of the pedestal 31.

In the embodiment, a guide hole 33 for receiving the longitudinal tube 22 to pass through is formed in one side of the basal body 32 of the base 30 facing the needle seat 10, and a receiving cavity 36 is formed below the guide hole 33.

In the embodiment, a receiving groove 34 is formed in the basal body 32 of the base 30, and clamping edges 35 are arranged on groove walls of both sides of the receiving groove 34.

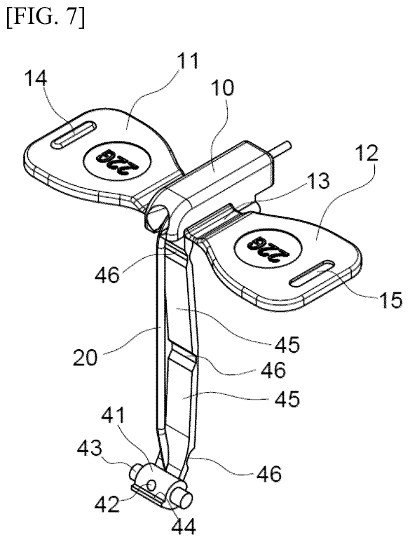

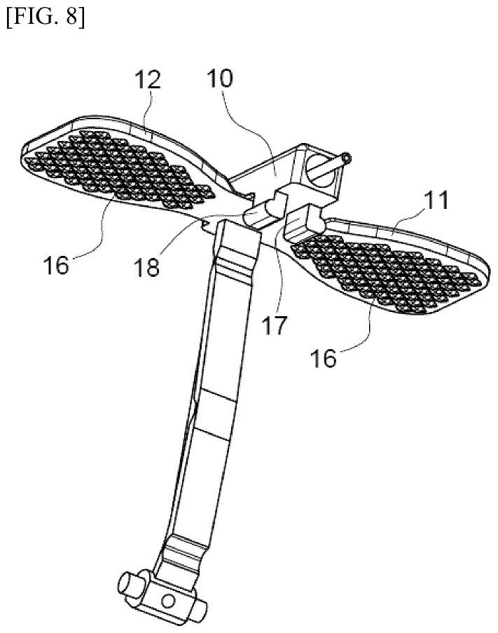

As shown in FIG. 7 and FIG. 8, the connecting member 40 in the embodiment includes a connecting base 41 and a foldable connector. One end of the connector is connected with the connecting base 41 and the other end of the connector is connected with the needle seat 10. The connector passes through the receiving groove 34 and is folded and unfolded in a region where the receiving groove 34 is located.

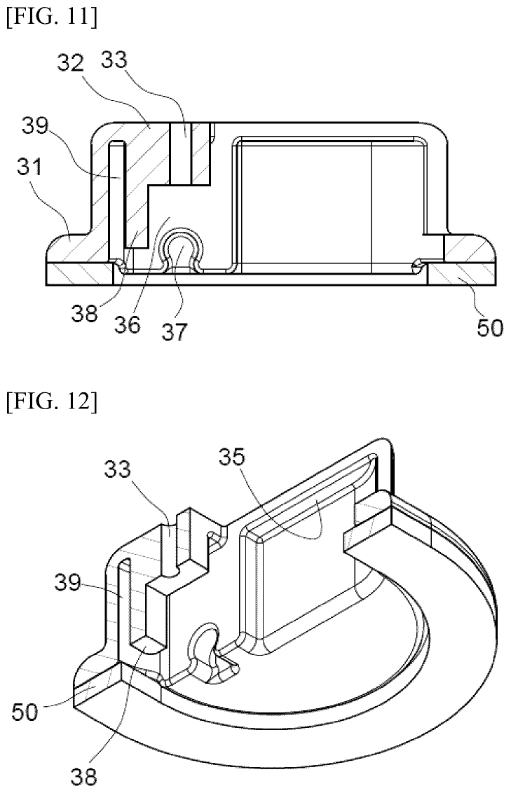

The connecting base 41 is located below the receiving cavity 36 and is rotationally and movably connected with the base 30. Preferably, hinge pins 43 are arranged at both ends of the connecting base 41. Correspondingly, as shown in FIG. 11 and FIG. 12, pin holes 37 adapted to the hinge pins 43 are formed in an inner wall of the receiving cavity 36 of the base 30.

The connector in the embodiment includes two connecting rods 45. To achieve folding and unfolding, flexible portions 46 are arranged between the needle seat 10 and the connecting rod 45, between adjacent connecting rods 45, and between the connecting rod 45 and the connecting base 41. The connector deforms at the flexible portions 46 to achieve state change between folding and unfolding.

It should be noted that the structure of the connector is a preferred mode of the embodiment. Equivalently, the flexible portion 46 may also be arranged as a hinge, and the connector may also be of a flexible material and can deform integrally.

As a special part of the embodiment, a connecting hole 42 for receiving the longitudinal tube 22 to pass through is formed in the connecting base 41. As shown in FIG. 1 and FIG. 2, when the connector is in the folded state and the safety needle assembly is in use, the longitudinal tube 22 passes through the guide hole 33, the receiving cavity 36, and the connecting hole 42 in sequence, and the tip of the needle tubing 20 stretches out below the base 30.

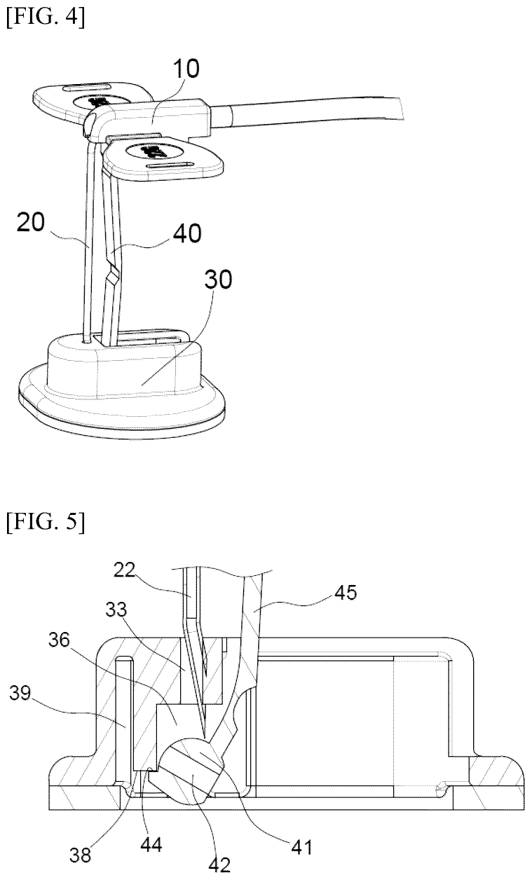

After the safety needle assembly is used, the needle seat 10 is stretched toward a direction away from the base 30 until the connector is in the unfolded state shown in FIG. 4 to FIG. 8. In this state, the tip of the longitudinal tube 22 is separated from the connecting hole 42 and is located in the receiving cavity 36. As shown in FIG. 5, when the tip of the longitudinal tube is separated from the connecting hole 42, the connector drives the connecting base 41 to rotate until the connecting hole 42 and the longitudinal tube 22 are in a staggered state. The tip of the longitudinal tube is restricted in the receiving cavity 36, and the tip of the longitudinal tube cannot enter the connecting hole 42 again, and cannot be exposed below the base 30 again. Therefore, the needle tip is automatically protected, and medical staff is prevented from being stabbed.

Further, to prevent the connecting base 41 from being reset after rotation, a non-return flange 44 is arranged on one side of the connecting base 41 in particular, and correspondingly, a non-return retaining arm 38 adapted to the non-return flange 44 is arranged on one side of the receiving cavity 36. As shown in FIG. 5, after the connecting base 41 rotates, the non-return flange 44 fits the non-return retaining arm 38, and the non-return retaining arm 38 prevents the connecting base 41 from rotating to reset, so as to guarantee the reliability of needle tip protection, and the safety is further improved.

Preferably, in the embodiment, as shown in FIG. 5, and FIG. 11 to FIG. 12, a gap 39 is arranged on one side of the non-return retaining arm 38 away from the connecting base 41. Generally, the base is made of an injection molded part which has certain deformability. After the gap 39 is arranged, in the process that the connecting base 41 rotates, the non-return flange 44 acts on one side of the non-return retaining arm 38 to drive the non-return retaining arm 38 to deform toward one side of the gap. The non-return retaining arm 38 is reset until the non-return flange 44 crosses the lower end of the non-return retaining arm 38 and fits the non-return flange 44 to prevent the connecting base from rotating reversely.

In the embodiment, the safety needle assembly is in the using state shown in FIG. 1 to FIG. 3. To prevent the positions between the needle seat 10 and the base 30 from changing easily, the clamping mechanism is arranged between the needle seat 10 and the base 30. When the connector is in the folded state, the needle seat 10 and the base 30 are connected together through the clamping mechanism.

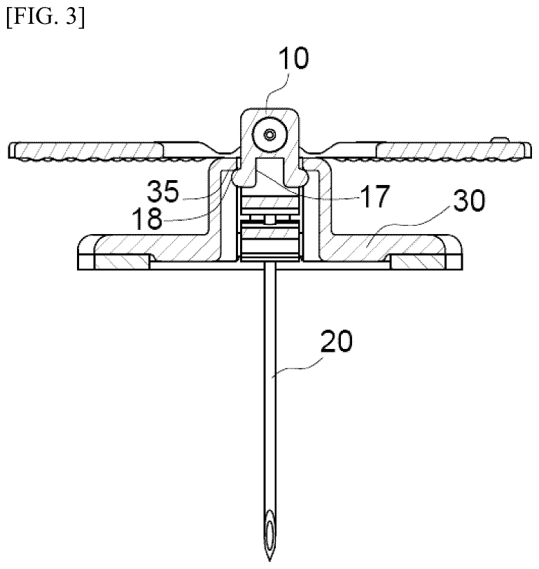

Preferably, the clamping mechanism in the embodiment, as shown in FIG. 3 and FIG. 8, includes a pair of clamping arms 17 extending toward one side of the base 30 from the bottom of the needle seat 10, and protrusions 18 protruding outward from the ends of free ends of the clamping arms 17. After the pair of clamping arms 17 stretch into the receiving groove 34, the pair of protrusions 18 fit the clamping edges 35, so that the needle seat and the base are in a relative fixed state in position.

After the use of the safety needle assembly is completed, when the needle seat stretches toward one side away from the base, the drawing force first drives the protrusions 18 and the clamping edges 35 to relieve fitting, so that the needle seat may displace toward one side away from the base.

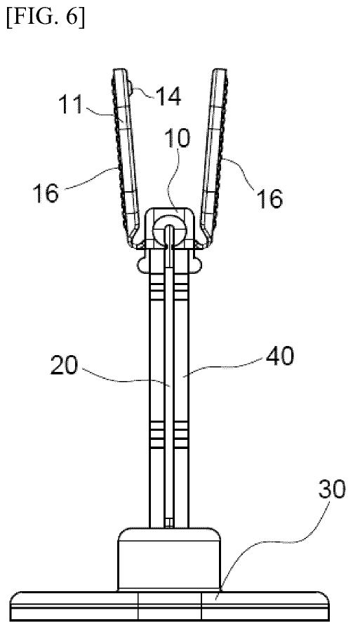

Preferably, as shown in FIG. 1 to FIG. 8, in the embodiment, wings are arranged on both sides of the needle seat 10, which are respectively a left wing 11 and a right wing 12. Bendable portions 13 are arranged between the left wing 11 and the needle seat and between the right wing 12 and the needle seat. As shown in FIG. 6, when the user drives the needle seat to move toward one side away from the base, the two wings are bent toward the middle to increase the force-applying area, so that the operation is more convenient.

As a preferred embodiment, as shown in FIG. 8, a damping portion 16 is arranged on one side of the wing facing the base 30. When the wing is in the state shown in FIG. 6, the damping portion 16 may increase the frictional force between the wing and fingers of the user to prevent a slipping phenomenon. The needle seat may be moved toward the side away from the base in a labor-saving and convenient manner.

As a preferred embodiment, as shown in FIG. 7, positioning bonding structures are arranged between the two wings, which are respectively a positioning bonding protrusion 14 arranged on the left wing 11 and a positioning bonding groove 15 arranged on the right wing 12. The positioning bonding protrusion and the positioning bonding groove correspond in position, and after the two wings are bent toward the middle, the two wings forms a whole through the cooperation of the positioning bonding protrusion and the positioning bonding groove.

Above all, what is described above is merely preferred embodiments of the present disclosure, but is not used for limiting the present disclosure. Any modifications, equivalent replacements, improvements and the like which are made within the spirit and principle of the present disclosure shall fall within the protection scope of the present disclosure.

1. A safety needle assembly, at least comprising:
a needle seat;
a needle tubing, wherein the needle tubing is bent to form a horizontal tube and a longitudinal tube, and the horizontal tube is fixedly connected with the needle seat;
a base, wherein a guide hole for receiving the longitudinal tube to pass through is formed in one side of the base facing the needle seat, and a receiving cavity is formed below the guide hole;
further comprising a connecting member wherein the connecting member comprises a connecting base and a foldable connector, the connecting base is located below the receiving cavity and is rotationally and movably connected with the base, one end of the connector is connected with the connecting base and the other end of the connector is connected with the needle seat; and a connecting hole for receiving the longitudinal tube to pass through is formed in the connecting base; and
the connector comprises a folded state and an unfolded state, in the folded state, the longitudinal tube passes through the guide hole, the receiving cavity, and the connecting hole in sequence; and in the unfolded state, the tip of the longitudinal tube is located in the receiving cavity, and the connecting base rotates until the connecting hole and the longitudinal tube are in a staggered state.

2. The safety needle assembly according to claim 1, wherein a non-return flange is arranged on one side of the connecting base, and a non-return retaining arm adapted to the non-return flange is arranged on one side of the receiving cavity.

3. The safety needle assembly according to claim 2, wherein a gap is arranged on one side of the non-return retaining arm away from the connecting base.

4. The safety needle assembly according to claim 1, wherein the connector comprises at least two connecting rods and flexible portions are arranged between the needle seat and the connecting rod, between adjacent connecting rods, and between the connecting rod and the connecting base.

5. The safety needle assembly according to claim 1, wherein hinge pins are arranged at both ends of the connecting base, and pin holes adapted to the hinge pins are formed in an inner wall of the receiving cavity of the base.

6. The safety needle assembly according to claim 1, wherein a clamping mechanism is arranged between the needle seat and the base, and when the connector is in the folded state, the needle seat and the base are connected together through the clamping mechanism.

7. The safety needle assembly according to claim 6, wherein the clamping mechanism comprises:
a pair of clamping arms extending toward one side of the base from the bottom of the needle seat;
protrusions protruding outward from the ends of free ends of the clamping arms; and
a receiving groove arranged on one side of the base facing the needle seat, wherein clamping edges adapted to the protrusions are arranged on groove walls of both sides of the receiving groove.

8. The safety needle assembly according to claim 1, wherein wings are arranged on both sides of the needle seat.

9. The safety needle assembly according to claim 8, wherein a bendable portion is arranged between the wing and the needle seat, and a damping portion is arranged on one side of the wing facing the base.

10. The safety needle assembly according to claim 9, wherein positioning bonding structures are arranged between the two wings, comprising a positioning bonding protrusion and a positioning bonding groove adapted to each other.

11. The safety needle assembly according to claim 2, wherein wings are arranged on both sides of the needle seat.

12. The safety needle assembly according to claim 11, wherein a bendable portion is arranged between the wing and the needle seat, and a damping portion is arranged on one side of the wing facing the base.

13. The safety needle assembly according to claim 12, wherein positioning bonding structures are arranged between the two wings, comprising a positioning bonding protrusion and a positioning bonding groove adapted to each other.

14. The safety needle assembly according to claim 3, wherein wings are arranged on both sides of the needle seat.

15. The safety needle assembly according to claim 14, wherein a bendable portion is arranged between the wing and the needle seat, and a damping portion is arranged on one side of the wing facing the base.

16. The safety needle assembly according to claim 15, wherein positioning bonding structures are arranged between the two wings, comprising a positioning bonding protrusion and a positioning bonding groove adapted to each other.

17. The safety needle assembly according to claim 4, wherein wings are arranged on both sides of the needle seat.

18. The safety needle assembly according to claim 17, wherein a bendable portion is arranged between the wing and the needle seat, and a damping portion is arranged on one side of the wing facing the base.

19. The safety needle assembly according to claim 18, wherein positioning bonding structures are arranged between the two wings, comprising a positioning bonding protrusion and a positioning bonding groove adapted to each other.

20. The safety needle assembly according to claim 5, wherein wings are arranged on both sides of the needle seat.

21. The safety needle assembly according to claim 20, wherein a bendable portion is arranged between the wing and the needle seat, and a damping portion is arranged on one side of the wing facing the base.

22. The safety needle assembly according to claim 21, wherein positioning bonding structures are arranged between the two wings, comprising a positioning bonding protrusion and a positioning bonding groove adapted to each other.

23. The safety needle assembly according to claim 6, wherein wings are arranged on both sides of the needle seat.

24. The safety needle assembly according to claim 23, wherein a bendable portion is arranged between the wing and the needle seat, and a damping portion is arranged on one side of the wing facing the base.

25. The safety needle assembly according to claim 24, wherein positioning bonding structures are arranged between the two wings, comprising a positioning bonding protrusion and a positioning bonding groove adapted to each other.

26. The safety needle assembly according to claim 7, wherein wings are arranged on both sides of the needle seat.

27. The safety needle assembly according to claim 26, wherein a bendable portion is arranged between the wing and the needle seat, and a damping portion is arranged on one side of the wing facing the base.

28. The safety needle assembly according to claim 27, wherein positioning bonding structures are arranged between the two wings, comprising a positioning bonding protrusion and a positioning bonding groove adapted to each other.
